(19) United States
(12) Patent Application Publication
(10) Pub. No.: US 2016/0120903 A1
(43) Pub. Date: May 5, 2016

(54) Topical Relief Cream Formulation for Allergic Reactions to Poison Ivy, Poison Oak, Poison Sumac, Minor Skin Irritations and Insect Bites

(71) Applicants: Raul M. Romero, Bartlett, TN (US); Van Jones, Memphis, TN (US)

(72) Inventors: Raul M. Romero, Bartlett, TN (US); Van Jones, Memphis, TN (US)

(21) Appl. No.: 14/748,959

(22) Filed: Jun. 24, 2015

Related U.S. Application Data
(60) Provisional application No. 62/069,936, filed on Oct. 29, 2014.

(51) Int. Cl. A61K 33/30; A61K 9/06; A61K 31/315; A61K 31/138; A61K 31/125; A61K 31/045; A61K 38/12; A61K 9/00; A61K 31/5375

(52) U.S. Cl. CPC A61K 33/30; A61K 9/0014; A61K 9/06; A61K 31/315; A61K 31/5375; A61K 31/125; A61K 31/045; A61K 38/12; A61K 31/138

(57) ABSTRACT

A topical relief cream formulation for allergic reactions to poison ivy, poison oak, poison sumac, minor skin irritations and insect bites through chemical means. The relief cream formulation includes a quantity of zinc acetate, a quantity of zinc oxide, a quantity of pramoxine, a quantity of camphor, and a quantity of menthol suspended in a topical lotion base. The aforementioned compounds provide several medicinal properties from analgesic, anesthetic, antiseptic, and astringent categories when applied locally to an area of skin affected by the oils found in poison ivy, poison oak, and poison sumac, as well as, minor skin irritations and insect bites.

The current application claims a priority to the U.S. Provisional Patent application Ser. No. 62/069,936 filed on Oct. 29, 2014.

### FIELD OF THE INVENTION

The present invention relates generally to a topical relief cream. More specifically, the present invention relates to a formulation of a topical relief cream to treat symptoms associated with allergic contact dermatitis, insect bites, and the like.

### BACKGROUND OF THE INVENTION

Many jobs and activities take place in an outdoor environment, where people make incidental contact with foliage and wildlife, including insects. Incidental contact with poison ivy, poison oak and poison sumac imparts urushiol oil onto a passerby. People who are allergic to this oil can develop a form of dermatitis. Urushiol-induced contact dermatitis, a form of allergic contact dermatitis, is a condition suffered by hundreds of millions of people each year who come into direct contact with plants of the genus Toxicodendron, which includes poison ivy, poison oak, and poison sumac. Contact with the urushiol oil contained in these plants produces allergic rashes characterized by skin redness, inflammation, and in severe cases, papules, vesicles, or blisters. Symptoms of the developed rashes include persistent itching, burning or stinging, pain, and infection if the affected area is not allowed to heal properly. Incidental contact with insects can result in being bitten by the insect as a form of self-defense or parasitism. Common insect bites produce symptoms of a similar itching or burning sensation to the urushiol oil contact dermatitis but to a milder extent. Further, some insect bites can produce local swelling, numbed or tingling sensations, or even pain.

Primary treatment involves washing exposed skin thoroughly with soap and water as soon as possible after exposure is discovered. Soap or detergent is necessary, as urushiol is an oil and does not readily dissolve with water alone. Common secondary treatments include applying over-the-counter topical corticosteroids, antipruritics, skin protectants, and antihistamines to alleviate the symptoms of a developed rash. Some formulations that combine active ingredients have been developed to mitigate multiple symptoms associated with allergic contact dermatitis and similar allergic reactions and skin irritations.

The present invention is a topical relief cream formulation for allergic reactions to poison ivy, poison oak, poison sumac, minor skin irritations and insect bites. The relief formulation is applied locally to the affected area such that chemicals within the composition are absorbed into the skin of and around the affected area in order to reduce or possibly eliminate symptoms of urushiol oil and insect bites.

### BRIEF DESCRIPTION OF THE DRAWINGS

FIG. 1 is a block diagram illustrating the ingredients of the present invention.

FIG. 2 is a block diagram illustrating the ingredients of an alternate embodiment of the present invention including a quantity of antibiotics and a quantity of antihistamines.

### DETAIL DESCRIPTIONS OF THE INVENTION

All illustrations of the drawings are for the purpose of describing selected versions of the present invention and are not intended to limit the scope of the present invention.

The present invention is a topical relief cream formulation for allergic reactions to poison ivy, poison oak, poison sumac, minor skin irritations and insect bites. The topical relief cream formulation is applied locally to the affected area such that chemical compounds within the composition are absorbed into the skin of and around the affected area in order to reduce or possibly eliminate symptoms of urushiol oil and insect bites.

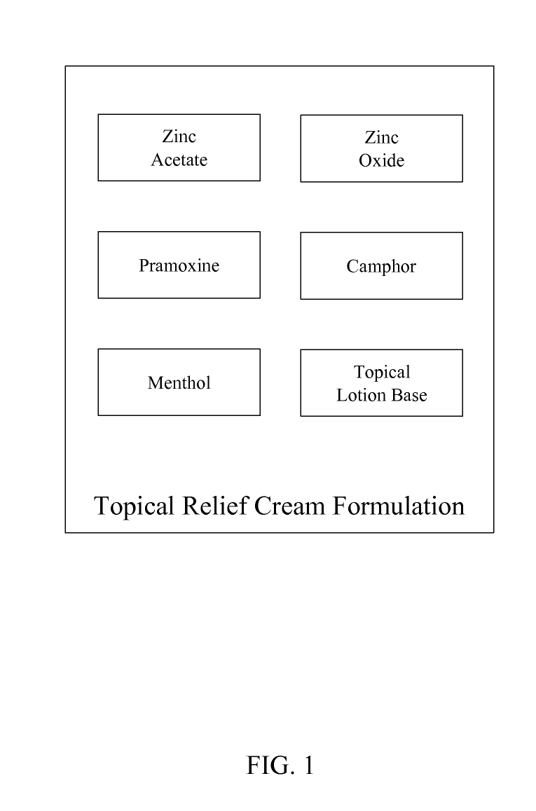

In accordance to FIG. 1, the present invention comprises a quantity of zinc acetate, a quantity of zinc oxide, a quantity of pramoxine, a quantity of camphor, a quantity of menthol, and a topical lotion base. The quantity of zinc acetate and the quantity of zinc oxide provide astringent properties to the relief cream formulation. The quantity of pramoxine and the quantity of camphor impart local anesthetic properties to the formulation. Additionally, the quantity of camphor provides antiseptic benefits to the present invention. The quantity of menthol imparts local analgesic properties to the present invention. The quantity of zinc acetate, the quantity of zinc oxide, the quantity of pramoxine, the quantity of camphor, and the quantity of menthol is homogeneously suspended within the topical lotion base, such that the quantity of zinc acetate, the quantity of zinc oxide, the quantity of pramoxine, the quantity of camphor, and the quantity of menthol are readily absorbed into the skin as the present invention is applied to the infected area.

As previously mentioned, the present invention imparts properties of astringents, anesthetics, analgesics, and antiseptics locally to the affected area. Astringents dry, harden, and protect the skin by binding. Astringents assist in relieving the rash and drying the oils from urushiol contact and insect bites. Anesthetics and analgesics relieve pain, specifically mitigating itching, stinging, and burning. Antiseptics reduce and prevent bacterial infection on the surface of the skin or within open wounds, which can result from scratching the affected area.

TABLE 1

| | Percent Weight of the Topical |
|---|---|
| Compound | Relief Cream Formulation |
| Zinc Acetate | 2.0% |
| Zinc Oxide | 12.0% |
| Pramoxine | 2.0% |
| Camphor | 0.5% |
| Menthol | 0.5% |

In accordance to Table 1 and the preferred embodiment of the present invention, the quantity of zinc acetate is approximately 2% by weight (wt) of the present invention and the quantity of zinc oxide is approximately 12% wt of the present invention. The quantity of pramoxine is approximately 2% wt of the present invention. Both of the quantity of camphor and the quantity of menthol are approximately 0.5% wt of the present invention. The composition of the preferred embodiment of the present invention allows the present invention to supply a sufficient amount of astringents, anesthetics, antiseptics, and analgesics to the infected area in order to assuage the symptoms of urushiol contact dermatitis and insect bites.

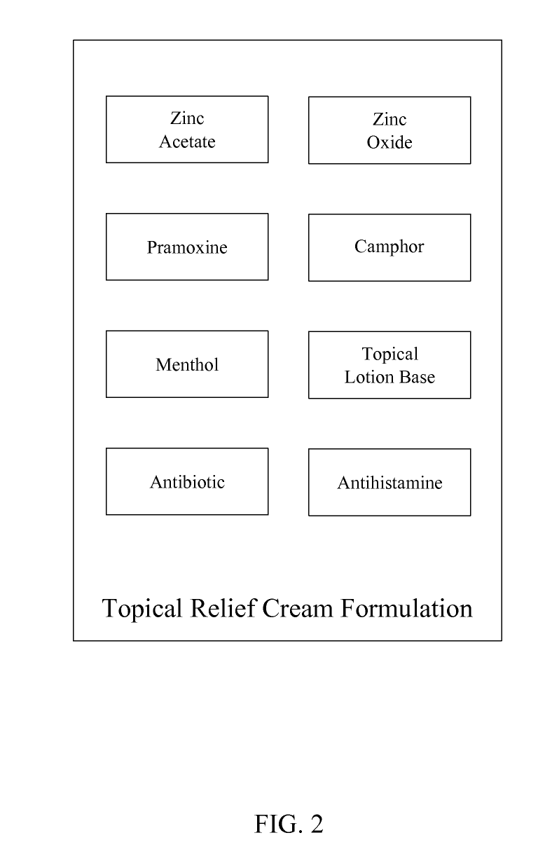

In some embodiments of the present invention, the present invention comprises a quantity of antibiotics and a quantity of antihistamines, in accordance to FIG. 2. The quantity of antibiotics is a topically prepared polypeptide for infection prevention, specifically, bacitracin. The quantity of antihistamines provides properties which assist in treating the allergic reaction resulting from contact with urushiol oil and from insect bites. The quantity of antihistamines is preferred to be diphenhydramine. The quantity of antibiotics and the quantity of antihistamines are homogeneously mixed into the relief cream formulation such that the quantity of antibiotics are readily available when the relief cream formulation is applied to an affected area.

In order to manufacture the present invention, the quantity of zinc acetate and the quantity of promoxine are dissolved into benzoyl alcohol, ethoxy diglycerol, or other low toxicity solvents, forming a primary preparatory mixture. Subsequently, the quantity of zinc oxide, the quantity of camphor, and the quantity of menthol are homogeneously mixed into the primary preparatory mixture forming a secondary preparatory mixture. The secondary preparatory mixture is then preferred to be wetted using glycerin as a smoothing agent to form a relief paste. Finally, the topical lotion base is added to the relief paste to form the topical relief cream formulation in accordance to the aforementioned composition, wherein the topical lotion base is a natural, moisturizing cream base including plant extracts which assist in moisturizing the affected area of skin.

The present invention is topically applied directly to the affected area by lightly massaging the topical relief cream formulation into the affected area. Once the topical relief cream formulation is applied, the quantity of zinc acetate, the quantity of zinc oxide, the quantity of pramoxine, the quantity of camphor, the quantity of menthol, the quantity of antibiotics, and the quantity of antihistamine are absorbed into the skin in order to impart their medicinal properties to the affected area. An increase of the effectiveness of the present invention is seen when the user first washes the affected area with soap and water and then dries the affected area before application.

Although the invention has been explained in relation to its preferred embodiment, it is to be understood that many other possible modifications and variations can be made without departing from the spirit and scope of the invention as hereinafter claimed.

What is claimed is:

1. A topical relief cream formulation for allergic reactions to poison ivy, poison oak, poison sumac, minor skin irritations and insect bites comprises:
a quantity of zinc acetate;
a quantity of zinc oxide;
a quantity of pramoxine;
a quantity of camphor;
a quantity of menthol;
a topical lotion base; and
the quantity of zinc acetate, the quantity of zinc oxide, the quantity of pramoxine, the quantity of camphor, and the quantity of menthol being homogeneously suspended within the topical lotion base.

2. The topical relief cream formulation for allergic reactions to poison ivy, poison oak, poison sumac, minor skin irritations and insect bites, as claimed in claim 1, comprises:
the quantity of zinc acetate being approximately 2% by weight of the topical relief cream formulation.

3. The topical relief cream formulation for allergic reactions to poison ivy, poison oak, poison sumac, minor skin irritations and insect bites, as claimed in claim 1, comprises:
the quantity of zinc oxide being approximately 12% by weight of the topical relief cream formulation.

4. The topical relief cream formulation for allergic reactions to poison ivy, poison oak, poison sumac, minor skin irritations and insect bites, as claimed in claim 1, comprises:
the quantity of pramoxine being approximately 2% by weight of the topical relief cream formulation.

5. The topical relief cream formulation for allergic reactions to poison ivy, poison oak, poison sumac, minor skin irritations and insect bites, as claimed in claim 1, comprises:
the quantity of camphor being approximately 0.5% by weight of the topical relief cream formulation.

6. The topical relief cream formulation for allergic reactions to poison ivy, poison oak, poison sumac, minor skin irritations and insect bites, as claimed in claim 1, comprises:
the quantity of menthol being approximately 0.5% by weight of the topical relief cream formulation.

7. The topical relief cream formulation for allergic reactions to poison ivy, poison oak, poison sumac, minor skin irritations and insect bites, as claimed in claim 1, comprises:
a quantity of antibiotics; and
the quantity of antibiotics being homogeneously mixed into the relief cream formulation.

8. The topical relief cream formulation for allergic reactions to poison ivy, poison oak, poison sumac, minor skin irritations and insect bites, as claimed in claim 7, comprises:
the quantity of antibiotics being bacitracin.

9. The topical relief cream formulation for allergic reactions to poison ivy, poison oak, poison sumac, minor skin irritations and insect bites, as claimed in claim 1, comprises:
a quantity of antihistamine; and
the quantity of antihistamine being homogeneously mixed into the relief cream formulation.

10. The topical relief cream formulation for allergic reactions to poison ivy, poison oak, poison sumac, minor skin irritations and insect bites, as claimed in claim 9, comprises:
the quantity of antihistamine being diphenhydramine.

11. A topical relief cream formulation for allergic reactions to poison ivy, poison oak, poison sumac, minor skin irritations and insect bites comprises:
a quantity of zinc acetate;
a quantity of zinc oxide;
a quantity of pramoxine;
a quantity of camphor;
a quantity of menthol;
a topical lotion base;
a quantity of antibiotics;
a quantity of antihistamine; and
the quantity of zinc acetate, the quantity of zinc oxide, the quantity of pramoxine, the quantity of camphor, the quantity of menthol, the quantity of antibiotics, and the quantity of antihistamine being homogeneously suspended within the topical lotion base.

12. The topical relief cream formulation for allergic reactions to poison ivy, poison oak, poison sumac, minor skin irritations and insect bites, as claimed in claim 11, comprises:
the quantity of zinc acetate being approximately 2% by weight of the topical relief cream formulation.

13. The topical relief cream formulation for allergic reactions to poison ivy, poison oak, poison sumac, minor skin irritations and insect bites, as claimed in claim 11, comprises:
the quantity of zinc oxide being approximately 12% by weight of the topical relief cream formulation.

14. The topical relief cream formulation for allergic reactions to poison ivy, poison oak, poison sumac, minor skin irritations and insect bites, as claimed in claim 11, comprises:
the quantity of pramoxine being approximately 2% by weight of the topical relief cream formulation.

15. The topical relief cream formulation for allergic reactions to poison ivy, poison oak, poison sumac, minor skin irritations and insect bites, as claimed in claim 11, comprises:
the quantity of camphor being approximately 0.5% by weight of the topical relief cream formulation.

16. The topical relief cream formulation for allergic reactions to poison ivy, poison oak, poison sumac, minor skin irritations and insect bites, as claimed in claim 11, comprises:
the quantity of menthol being approximately 0.5% by weight of the topical relief cream formulation.

17. The topical relief cream formulation for allergic reactions to poison ivy, poison oak, poison sumac, minor skin irritations and insect bites, as claimed in claim 11, comprises:
the quantity of antibiotics being bacitracin.

18. The topical relief cream formulation for allergic reactions to poison ivy, poison oak, poison sumac, minor skin irritations and insect bites, as claimed in claim 11, comprises:
the quantity of antihistamine being diphenhydramine.
